(19) United States
(12) Patent Application Publication
(10) Pub. No.: US 2018/0015258 A1
(43) Pub. Date: Jan. 18, 2018

(54) Cover for a peripherally inserted central catheter (PICC) line

(71) Applicant: Hsiu Chin Shen, Shelby Township, MI (US)

(72) Inventor: Hsiu Chin Shen, Shelby Township, MI (US)

(21) Appl. No.: 15/145,745

(22) Filed: Jul. 14, 2016

(51) Int. Cl. A61M 25/02

(52) U.S. Cl. CPC A61M 25/02; A61M 2205/59; A61M 2025/0206; A61M 2205/0205; A61M 2205/0216

(57) ABSTRACT

A protective cover for use with peripheral inserted central catheters (PICC) to secure the outer portion of the catheter lines. The device is an elasticated sleeve so as to snugly fit over the extremity that has the exposed peripheral catheter lines.

### FIELD OF INVENTION

The field relates to covers for exposed medically inserted tubing. For patients needing intravenous infusions over short-term and long-term these tubes are used for the delivery of the fluids.

### BACKGROUND

Patients sometimes have an ongoing need for administration of medication by Intravenous (IV) therapy. Many forms of IV therapy exist, but for long-term use a Peripherally Inserted Central Catheter (PICC) line may be one means of delivering IV fluids and medication. A PICC line is inserted into a peripheral vein, often in the arm, then threaded until the catheter is in the superior vena cava of the heart.

IV lines sometimes include dangling bags and therefore support is often needed. Waterproof polymeric materials have been used for protective covers as described in Matyas, U.S. Pat. No. 5,885,254 and Warner, U.S. Pat. No. 6,276,364. However, many patients, especially younger patients find such covers are aesthetically unappealing.

Self-supporting fabric covers are a little more appealing in appearance. Such covers are problematic however, being typically secured by fasteners, they may restrict movement. Examples of such covers are shown in patents by Harris, U.S. Pat. No. 6,540,724 and Villiapiano, U.S. Pat. No. 6,032,289.

A cover is needed that provides easy access to the intravenous tubing, that is easy to install and remove, that may be worn continuously, that does not restrict movement, and also that is aesthetically pleasing to wear.

### BRIEF SUMMARY OF THE INVENTION

A sleeve secures the PICC lines needed for use over the short-term and long-term. The sleeve is tubular, slips over the limb, and has no need of any fasteners. The tubular cuff is formed from a length of material or by joining two longitudinal edges of a sheet of material into a tubular form.

The tubular cuff is stretchable, easily slipping onto the limb and remaining fitted in place by tensile forces of the resilient material. The gentle tension allows the cuff to remain in place but provides access to the lines needed for IV therapy.

The objective of the cuff is to protect the intravenous lines and related attachments during normal daily activities, without restricting freedom of movement of the limb. The cuff must also allow easy access to the intravenous lines for intravenous infusions.

One objective of the cuff is to provide better aesthetics and comfort to patients, including children. Still another object of the cuff is to allow for the IV tubing and related attachments to be protected during and between treatments without restricting freedom of movement of the patient.

### BRIEF DESCRIPTION OF THE DRAWINGS

FIG. 1 illustrates an example of the features of a PICC line cover. Feature 101 is the hole through which the PICC lines will pass. Feature 102 is the end that will cover the lines during use of the cover. Feature 103 is the end of the cover that will fold over the complete assembly.

FIG. 2 illustrates an example of a PICC Line Cover illustrating the infusion lines passing through the hole in the cover.

FIG. 3 illustrates the PICC Line Cover in use, initially covering the infusion lines being covered by end 102 of the cover. During normal use end 102 would completely cover the exposed PICC lines. Feature 50 illustrates the ends of the PICC lines before being completely covered by the cover.

FIG. 4 illustrates the PICC Line Cover in use showing the completed double covering of the infusion lines

### DETAILED DESCRIPTION OF THE INVENTION

The examples described and the drawings rendered are illustrative and are not to be read as limiting the scope of the invention as it is defined by the appended claims.

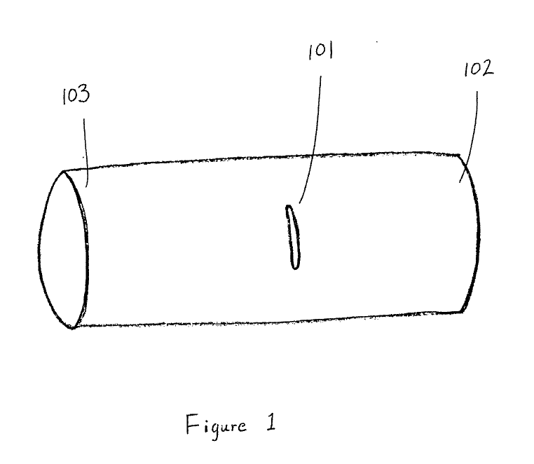

Illustrated in FIG. 1 is a cover for use on a patient's limb and is made of a resilient, flexible, and breathable material formed into a tube. The circumference of the tube and the tensile properties of the stretchable material provides a snug fit on the limb of a patient. The elasticity of the material helps to keep the cover fitted to the limb and in a fixed position on the limb without significantly restricting movement. As long as the material has the desired elasticity and resilience such materials may have the appearance of any fabric such as cotton, nylon, polyester, or acrylic, thereby matching other clothing the patient might be wearing.

The collection of perspiration under the cover would creating favorable conditions to exacerbate various skin infections and skin conditions. The cover must be made of material that is breathable so as not to encourage collection of perspiration.

For best use the hole 101 should not be in the middle, with respect to the length of the cover, with end 103, containing the longer length, closer to the medial part of the body.

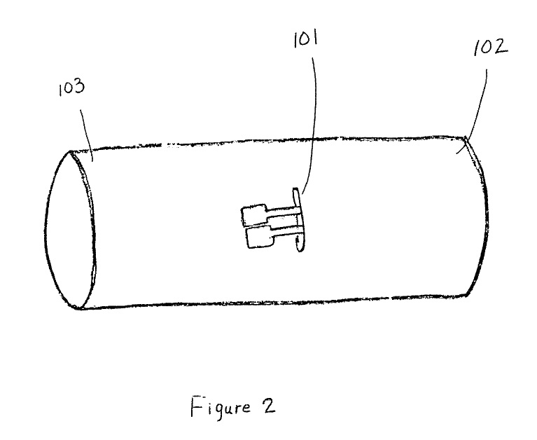

Illustrated in FIG. 2 is a cover for use on a patient's limb and shows the intravenous lines passing through hole 101. In this manner the lines do not come in direct contact with the dressing nor in direct contact with the skin. This would reduce the potential for chaffing and reduce the potential for irritation from the contact for patients with sensitive skin.

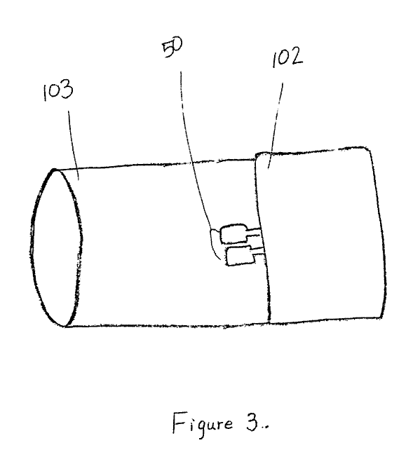

Illustrated in FIG. 3 is a cover in use on a patient's limb, and shows end 102 covering the intravenous lines 50. In normal use end 102 would completely cover the lines 50, and lines 50 would not be in any way exposed.

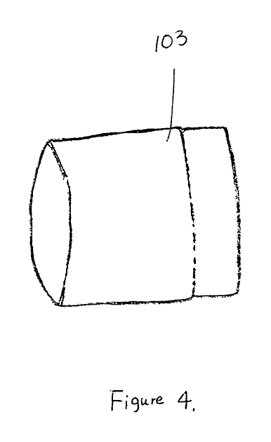

Illustrated in FIG. 4 is a cover in use on a patient's limb, and shows end 103 folding down to completely double cover the intravenous lines and end 102. In normal use end 103 would fold down past the fold of end 102 to completely enclose both.

The cover may include antimicrobial properties, by additives such as that provided by Purthread®, and Microban®, thereby reducing the opportunity for growth of infection causing microorganisms. Purthread is a registered trademark of PurThread Technologies, Inc., http://www.purthread.com/. Microban® is a registered trademark of Microban Products Company. http://www.microban.com/

The cover may be sized to fit patients of different ages and sizes. The tubular cover in FIG. 1 is sized less than the circumference of the limb such that tensile forces holds the cover in place without substantially restricting movement or circulation.

An intravenous site may contain lines, and a mechanism for connecting to lines and attachments. Attachments may include an attachment for injection of medications. The elasticity of the illustrated cover is such that it conceals these additional attachments. FIG. 2 shows a PICC line with an attachment mechanism is concealed by the a section of the PICC line cover.

The hole in the PICC Line cover may be positioned lengthwise in the middle, or off center. It is preferable to be positioned off center. In this manner the short section is used first to cover the lines, then the long section has enough length to completely double cover the lines and bottom part of the cover.

A cover may be worn over the upper extremity of the arm, above the elbow region or elsewhere on an arm or leg, so long as it is properly sized. A cover may even be worn on a torso, such as a tube top, as an item of clothing, if properly sized. The cover may be made of materials suitable for clothing and may be worn like any clothing.

In the examples of FIGS. 3 and 4 additional step in the usage are illustrated. The slot 101 such as illustrated in FIGS. 3 and 4 is not in the middle of the long axis of the cover. The slot is such that an IV tube extending from a patient's arm may be passed through the slot 101. The cover is worn such that the long end is closest to the medial part of the limb, and the tubes pass through the slot. The lower end then is pulled over the lines thus concealing them. The top part of the cover then folds down and over the lines, thereby providing double protection and double assurance the lines will stay in place. This totality of protect is such that no restriction of motion occurs.

FIGS. 1, 2, 3, and 4 illustrate an example of a resilient, flexible cover. The elasticated resilience of the material allows the cover, when properly sized to firmly but comfortably fit on a limb. The cover will remain in place, without significantly restricting circulation or freedom of movement

Alternative combinations and variations of the examples provided will become apparent based on this disclosure. It is not possible to provide specific examples for all of the many possible combinations and variations of the embodiments described, but such combinations and variations may be claims that eventually issue.

What is claimed is:

1. A device for use with an PICC line, tubing, and associated dressing for patients undergoing treatments comprising: a tubular cover being made of a material capable of concealing the presence of the intravenous lines and associated dressings when fitted on a limb.

2. The device of claim 1, wherein the cover has a slot to allow access to the intravenous lines and associated dressing during intravenous infusion treatment.

3. The device of claim 2, wherein the cover forms a closed tube without a fastener.

4. The device of claim 3, wherein the cover encloses the intravenous lines and associated dressing.

5. The device of claim 4, wherein the cover helps keep the intravenous lines and associated dressing from being soiled during normal daily activities.

6. The device of claim 5, wherein the cover is sized, such that tension in the material does not substantially restrict the movement or circulation in the limb.

7. The device of claim 6, whereby during intravenous infusion treatments instead of removing the cover, it is capable of being left on the limb.

8. The device of claim 7, whereby during changing of the dressing instead of removing the cover, it is capable of being left on the limb

9. The device of claim 8, wherein the cover forms a double protection after proper installation.

10. The device of claim 9, wherein the material of the cover is stretchable, such that the cover is capable of being slipped onto a limb, remaining fitted onto the limb by the elasticity of the stretchable material.

11. The device of claim 10, wherein the material of the cover is breathable, such that perspiration does not accumulate on the skin, thereby reducing the opportunity for adverse skin conditions.

12. The device of claim 11, wherein the material of the cover may be infused with antimicrobial properties, thereby reducing the opportunity for PICC line infections.

13. The device of claim 12, wherein contains a slot allowing storage of the intravenous lines during normal daily activities.

14. The device or claim 13 wherein from one end of the cover to the other the position of the slot is not in the center.

15. The device of claim 14, wherein the intravenous lines go through the slot, separating the intravenous lines from contact with the skin thereby reducing the potential for chaffing.
